(19) United States
(12) Patent Application Publication
(10) Pub. No.: US 2013/0281782 A1
(43) Pub. Date: Oct. 24, 2013

(54) COMBINED RIGID URETEROSCOPE

(71) Applicant: Junhong Zhou, Guangdong (CN)

(72) Inventor: Junhong Zhou, Guangdong (CN)

(21) Appl. No.: 13/977,721

(22) PCT Filed: Jan. 10, 2012
(86) PCT No.: PCT/CN12/70201

§ 371 Date: Jun. 30, 2013

(30) Foreign Application Priority Data
Jan. 28, 2011 (CN) 201110030512.9

(51) Int. Cl. A61B 1/307; A61B 1/00

(52) U.S. Cl. CPC A61B 1/307; A61B 1/00135; A61B 1/00078
USPC 600/125

(57) ABSTRACT

Disclosed is a combined rigid ureteroscope which comprises a rigid ureteroscope body and a tubular sheath outwardly fitted on a tubular part of the ureteroscope body. A starting end of the sheath is closely engaged with the tubular part of the ureteroscope body. A locking mechanism is provided at a rear end of the sheath. A locking part is provided between the tubular part of the ureteroscope body and an operation part of the ureteroscope body. The locking mechanism is cooperated with the locking part to lock or unlock the rigid ureteroscope body and the tubular sheath. In operations of examining, diagnosing and treating the ureter and the kidney by employing this invention, it saves operation procedures, shortens operation time, improves operation safety, avoids problems of difficult operation and easy damage of the flexible ureteroscope, and realizes safety, effectiveness and low cost in the clinical treatment.

### FIELD OF TECHNOLOGY

The present invention relates to a medical device, and particularly, to a combined rigid ureteroscope.

### BACKGROUND

An ureteroscope, which is an endoscope for examination, diagnosis and treatment of ureter and kidney, comprises a flexible ureteroscope and a rigid ureteroscope, which may be respectively suitable for diagnosis and treatment of different parts of upper urinary tract according to their characteristics. When examination and lithotrity are performed with a structure of an existing ureteroscope, stones need to be taken out from human body through a cavity of the ureteroscope after being crushed by a lithotriptic device. Due to the small cavity, slightly larger crushed stones can not pass through, which are merely taken out from the human body together with the ureteroscope. Therefore, the ureteroscope needs to get into and out of the urethra, the bladder and the ureter even the kidney repeatedly for taking out crushed stones, resulting increased operation time and operation risks, and multiple insertion and removal also bring patients more pains. In another aspect, when diseases of the renal pelvis, the renal calyx and the upper ureter are diagnosed and treated by employing the flexible ureteroscope, the ureter orifice needs to be dilated and examined by the rigid ureteroscope firstly, and a guide wire is left, and then the rigid ureteroscope is taken out, and a hose is placed into the ureter along the guide wire, then the guide wire is taken out, and the flexible ureteroscope is placed along the hose for the diagnosis and treatment of the diseases of the upper ureter, the renal pelvis and the renal calyx. Since the hose can not be placed under watching in the whole operation, it will increase risks of damaging the ureter and results cumbersome and complex operations. Furthermore, the flexible ureteroscope is expensive, difficult to operate and susceptible to damage, and its frequent use will increase operation time and operation cost, which is unfavorable to popularize and develop. Therefore, the structure design of the existing ureteroscope can not meet the requirements of safety, effectiveness and low cost in modern clinical treatment.

### SUMMARY OF THE INVENTION

To solve the above technical problems, the present invention provides a combined rigid ureteroscope.

The technical solutions employed by the present invention for the solution of its problems are:

A combined rigid ureteroscope comprises a rigid ureteroscope body and a tubular sheath outwardly fitted on a tubular part of the ureteroscope body, a starting end of the sheath being closely engaged with the tubular part of the ureteroscope body, a locking mechanism being provided at a rear end of the sheath, a locking part being provided between the tubular part of the ureteroscope body and an operation part of the ureteroscope body, and the locking mechanism being cooperated with the locking part to lock or unlock the rigid ureteroscope body and the tubular sheath.

As a preferred embodiment of the present invention, the locking mechanism at the rear end of the sheath comprises a sleeve with an end cap and a pin fixed on a side wall of the sleeve, a slant chute being provided in an outer wall of the rear end of the tubular sheath, and an end of the pin extending into the chute; the locking part of the ureteroscope body being a ringed rib provided on a wall of the ureteroscope body; the ringed rib being snapped into the end cap of the sleeve, and the tubular sheath and the ureteroscope body being locked or unlocked with a movement of the pin in the chute.

As a further improvement of the present invention, the tubular sheath is a tapered tube, the starting end of the tubular part of the ureteroscope body extending out of the sheath, an outlet channel being formed by a gap between the wall of the sheath and the tubular part of the ureteroscope body, an inlet hole being provided in an outer wall of a front end of the sheath, and an outlet hole being provided in an outer wall of the rear end thereof.

The effects of the present invention lie in that the combined rigid ureteroscope of the present invention has following advantages in operations of examination, diagnosis and treatment of the ureter and the kidney:

A. the locking mechanism between the ureteroscope body and the sheath may be unlocked, and the rigid ureteroscope body and the sheath are separated, then the sheath is left in the ureter as a channel through which crushed stones are taken out repeatedly or the ureteroscope body gets into and out of the ureter independently, which shortens operation time, improves operation safety and eases pains of patients;

B. when diseases of renal pelvis, renal calyx and upper ureter are diagnosed and treated by employing the flexible ureteroscope, the sheath may be left directly as a channel through which the flexible ureteroscope gets in and out after the ureter orifice is dilated and examined by the combined rigid ureteroscope, without the guide wire and the hose being placed, which saves operating procedures. Furthermore, the procedure of leaving the sheath is completed under watching in the whole operation by means of an ocular of the rigid ureteroscope, which greatly improves safety compared to placement of the guide wire and the hose previously;

C. in procedures of the operation with the flexible ureteroscope, the rigid ureteroscope may be a replacement in some operating steps. The flexible and rigid ureteroscope may be alternately placed to operate via the sheath directly, thereby achieving ingenious cooperation of the flexible ureteroscope and the rigid ureteroscope, reducing the use of the flexible ureteroscope, avoiding problems of difficult operation and susceptible damage of the flexible ureteroscope, and indeed realizing safety, effectiveness and low cost in the clinical treatment.

D. when an outlet channel is formed between the wall of the sheath and the tubular part of the ureteroscope body and an inlet and an outlet are designed in the sheath, the washing fluid can flow from the channel, which overcomes the problem that continuous inlet and outlet of water can not be realized due to only one inlet and outlet channel in the conventional ureteroscope, thereby satisfying continuous rinsing by washing fluid in the operations, and avoiding increase of operation time and excess intraluminal pressure caused by alternation inlet and outlet of water while ensuring clear operation view, and greatly reducing risks of damaging the ureter and the kidney in the operations.

### BRIEF DESCRIPTION OF THE DRAWINGS

The present invention is further described in combination with accompanying drawings and embodiments below.

FIG. 1 is a schematic diagram of a whole structure of an embodiment of the present invention;

FIG. 2 is an exploded schematic diagram of FIG. 1;

FIG. 3 is an exploded schematic diagram of another embodiment of the present invention.

### EMBODIMENTS OF THE INVENTION

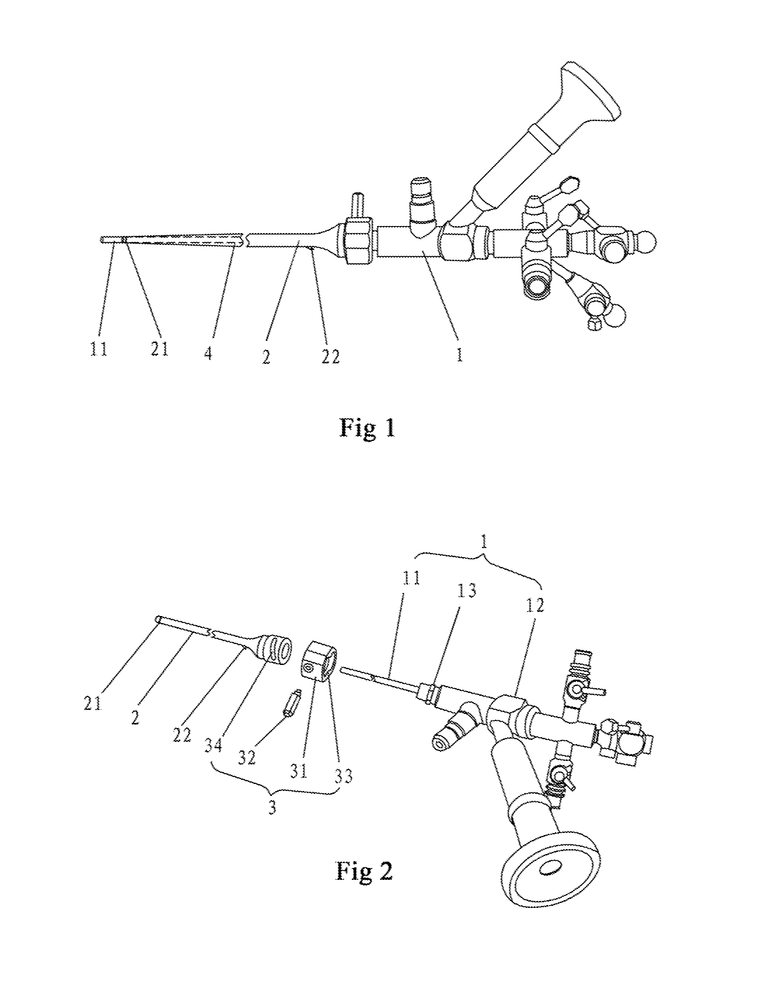

With reference to FIG. 1 and FIG. 2, a combined rigid ureteroscope comprises a rigid ureteroscope body 1 and a tubular sheath 2 outwardly fitted on a tubular part 11 of the ureteroscope body. A starting end of the sheath 2 is closely engaged with the tubular part 11 of the ureteroscope body. A locking mechanism 3 is provided at a rear end of the sheath 2, and a locking part 13 is provided between the tubular part 11 of the ureteroscope body and an operation part 12 of the ureteroscope body, and the locking mechanism 3 is cooperated with the locking part 13 to lock or unlock the rigid ureteroscope body 1 and the tubular sheath 2.

In the present embodiment, the locking mechanism 3 at the rear end of the sheath 2 comprises a sleeve 31 with an end cap 33 and a pin 32 fixed on a side wall of the sleeve 31. A slant chute 34 is provided in an outer wall of the rear end of the tubular sheath 2, and an end of the pin 32 extends into the chute 34. The locking part 13 of the ureteroscope body 1 is a ringed rib provided on a wall of the ureteroscope body; the ringed rib is snapped into the end cap 33 of the sleeve, and the tubular sheath 2 and the ureteroscope body 1 are locked or unlocked with a movement of the pin 32 in the chute 34.

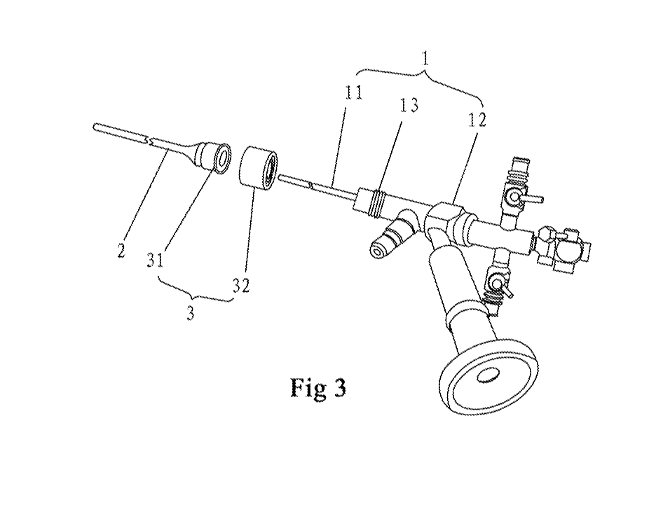

With reference to FIG. 3, in the present embodiment, the locking mechanism 3 at the rear end of the tubular sheath 2 is a threaded sleeve 32 fitted on a lug boss 31 of the rear end of the sheath; the locking part 13 of the ureteroscope body 1 is external threads provided on a wall thereof; the threaded sleeve 32 moves on the external threads to lock or unlock the tubular sheath 2 and the ureteroscope body 1.

When operations of examination, diagnosis and treatment of the ureter and the kidney are performed by employing the combined rigid ureteroscope of the present invention, the locking mechanism between the ureteroscope body and the sheath may be unlocked, and the rigid ureteroscope body and the sheath are separated, and the sheath is left in the ureter as a channel through which crushed stones are taken out repeatedly or the ureteroscope body gets into and out of the ureter independently, which shortens operation time, improves operation safety and eases pains of patients. When diseases of the renal pelvis, the renal calyx and the upper ureter are diagnosed and treated by employing the flexible ureteroscope, the sheath may be left directly as a channel through which the flexible ureteroscope gets in and out after the ureter orifice is dilated and examined by the combined rigid ureteroscope, without the guide wire and the hose being placed, which saves operating procedures. Furthermore, the procedure of leaving the sheath is completed under watching in the whole operation by means of an ocular of the rigid ureteroscope, which greatly improves safety compared to placement of the guide wire and the hose previously.

In procedures of the operation with the flexible ureteroscope, the rigid ureteroscope may be a replacement in some operating steps. For example, when stones of the renal calyx are crushed by employing the flexible ureteroscope, some crushed stones moves to the renal pelvis. Previously, the flexible ureteroscope merely continues to be used for taking out or crushing the stones. However, with the flexible ureteroscope, great difficulty to take out the stones, inefficiency to crush the stones and time consuming procedures all increase operation time and operation risks. By employing the combined rigid ureteroscope of the present invention, the flexible and rigid ureteroscope may be alternately placed to operate via the sheath left in the ureter directly, which is convenient to take out stones and fast to crush stones, thereby greatly reducing operation time and the use of the flexible ureteroscope, avoiding problems of difficult operation and easy damage of the flexible ureteroscope, and indeed realizing safety, effectiveness and low cost in the clinical treatment.

As a further improvement of the present invention, in the embodiments of FIG. 1 and FIG. 2, the tubular sheath 2 is a tapered tube, the starting end of the tubular part 11 of the ureteroscope body extends out of the sheath 2, an outlet channel 4 is formed by a gap between the wall of the sheath 2 and the tubular part 11 of the ureteroscope body, an inlet hole 21 is provided in an outer wall of a front end of the sheath, and an outlet hole 22 is provided in an outer wall of the rear end thereof. An outlet channel is added into the sheath in this improvement, which overcomes the problem that continuous inlet and outlet of water can not be realized due to only one inlet and outlet channel in the conventional ureteroscope, thereby satisfying continuous rinsing by washing fluid in the operations, and avoiding an increase of operation time and excess intraluminal pressure caused by alternation of inlet and outlet of water while ensuring clear operation view, and greatly reducing risks of damaging the ureter and the kidney in the operations.

The present invention can be widely applied to operations of examination, diagnosis and treatment of the ureter and the kidney.

1. A combined rigid ureteroscope, characterized in that, the combined rigid ureteroscope comprises a rigid ureteroscope body and a tubular sheath outwardly fitted on a tubular part of the ureteroscope body, a starting end of the sheath being closely engaged with the tubular part of the ureteroscope body, a locking mechanism being provided at a rear end of the sheath, a locking part being provided between the tubular part of the ureteroscope body and an operation part of the ureteroscope body, and the locking mechanism being cooperated with the locking part to lock or unlock the rigid ureteroscope body and the tubular sheath.

2. The combined rigid ureteroscope according to claim 1, characterized in that, the locking mechanism at the rear end of the sheath comprises a sleeve with an end cap and a pin fixed on a side wall of the sleeve, a slant chute being provided in an outer wall of the rear end of the tubular sheath, and an end of the pin extending into the chute; the locking part of the ureteroscope body being a ringed rib provided on a wall of the ureteroscope body; the ringed rib being snapped into the end cap of the sleeve, and the tubular sheath and the ureteroscope body being locked or unlocked with a movement of the pin in the chute.

3. The combined rigid ureteroscope according to claim 1, characterized in that, the locking mechanism at the rear end of the tubular sheath being a threaded sleeve fitted on a lug boss of the rear end of the sheath; the locking part of the ureteroscope body being external threads provided on a wall thereof; the threaded sleeve moving on the external threads to lock or unlock the tubular sheath and the ureteroscope body.

4. The combined rigid ureteroscope according to claim 1, characterized in that, the tubular sheath is a tapered tube, the starting end of the tubular part of the ureteroscope body extending out of the sheath, an outlet channel being formed by a gap between the wall of the sheath and the tubular part of the ureteroscope body, an inlet hole being provided in an outer wall of a front end of the sheath, and an outlet hole being provided in an outer wall of the rear end thereof.

5. The combined rigid ureteroscope according to claim 2, characterized in that, the tubular sheath is a tapered tube, the starting end of the tubular part of the ureteroscope body extending out of the sheath, an outlet channel being formed by a gap between the wall of the sheath and the tubular part of the ureteroscope body, an inlet hole being provided in an outer wall of a front end of the sheath, and an outlet hole being provided in an outer wall of the rear end thereof.

6. The combined rigid ureteroscope according to claim 3, characterized in that, the tubular sheath is a tapered tube, the starting end of the tubular part of the ureteroscope body extending out of the sheath, an outlet channel being formed by a gap between the wall of the sheath and the tubular part of the ureteroscope body, an inlet hole being provided in an outer wall of a front end of the sheath, and an outlet hole being provided in an outer wall of the rear end thereof.
